(19) United States
(12) Patent Application Publication
(10) Pub. No.: US 2007/0071779 A1
(43) Pub. Date: Mar. 29, 2007

(54) Compositions for delivering lipophilic agents to the intestinal mucosa and method of making thereof

(75) Inventor: Robert Allan McKie, Boise, ID (US)

Correspondence Address: ADVANTIA LAW GROUP, 9035 SOUTH 1300 EAST, SUITE 200, SANDY, UT, 84094, US

(73) Assignee: LEGGIT INGENUITY, LLC

(21) Appl. No.: 11/418,915

(22) Filed: May 4, 2006

Related U.S. Application Data
(60) Provisional application No. 60/720,380, filed on Sep. 26, 2005.

(51) Int. Cl. A61K 9/00; A61K 38/43; A61K 31/355; A61K 31/19; A61K 31/015

(52) U.S. Cl. 424/400; 424/94.1; 514/557; 514/458; 514/763

(57) ABSTRACT

A composition for delivering lipophilic agents to intestinal mucosa and method of making and use thereof are disclosed. The composition comprises an aqueous solution of one or more short chained C₂ to C₆ fatty acids, and corresponding salts thereof, and one or more lipophilic agents dissolved in the aqueous solution. Preferably the solution is buffered at a pH of between about 3.5 and 10.5. When the composition is administered to the colon and rectum, the short-chained fatty acids are consumed by the intestinal mucosa thereby forcing said lipophilic agents to enter the mucosal lipophilic cell membrane, thereby protecting the cells from the adverse effects of radiation and chemotherapy.

### PRIORITY CLAIM

Priority of U.S. Provisional patent application Ser. No. 60/720,380 filed on Sep. 26, 2005 is claimed.

### BACKGROUND

1. Field of the Invention

The present invention relates generally to compositions and a method for protecting the intestinal tract from the adverse effects of chemical or radiation therapy. More particularly, the present invention relates to a composition for delivering lipophilic antioxidants to intestinal mucosa and a method of making and using thereof.

2. Related Art

Currently, the intestinal lining is protected from damage during chemo and radiation therapy in a number of ways. The most important step is limiting the dose of chemotherapy and radiation therapy as much as possible. This limits damage to healthy tissue, but also limits the efficacy of cancer therapy. In the case of radiation therapy, limiting the dose of radiation therapy received by the intestine, and the volume of intestine irradiated is key. Amifostine is a currently available agent for prevention of radiation and chemotherapy induced damage to the intestinal tract. It is administered intravenously or subcutaneously, and it reduces the risk of radiation-induced damage to the rectum (radiation proctitis). Unfortunately, even with amifostine therapy, many individuals will develop radiation proctitis during or after radiation therapy. Amifostine requires intravenous or subcutaneous administration, and can induce hypotension and severe allergic reactions.

Treatment of radiation and chemotherapy-induced damage to the intestines is multi-faceted. Enemas of short chained-fatty acids, steroid enemas, laser therapy and even bowel resection with diverting colostomy have been utilized. Antioxidants, including amifostine and related compounds, are effective in treating or protecting mucosal tissue from damage associated with radiation and/or chemotherapeutic treatments.

### SUMMARY OF THE INVENTION

It has been recognized that it would be advantageous to develop a system for protecting the intestinal tract during disease therapy. Specifically, damage to the intestines occurs during radiation and chemotherapy for treatment of a number of tumors, and during the course of treatment for many other illnesses as well.

The invention provides a composition for delivering fat soluble chemicals to the intestinal lining in a water-based solution. The water-based composition of the present invention can be delivered to the intestines through feeding tubes, enemas and other delivery systems. The present composition for delivering lipophilic antioxidants to intestinal mucosa comprises a water solution in which short chained fatty acids are dissolved. These short chained fatty acids allow hydrophobic agents such as lipophilic antioxidants to be dissolved or suspended in an aqueous medium (emulsification). The hydrophobic agents suitable for the present invention can be lipophilic anti-oxidants and lipophilic drugs. Because the colon consumes short chained fatty acids as an energy source, the lipophilic anti-oxidants and drugs contained in the composition of the present invention become progressively less and less soluble in the aqueous medium. Therefore, when the composition of the present invention is administered to the colon, the hydrophobic anti-oxidants and drugs enter the lipophilic cell membrane driven by the intestinal consumption of the short chained fatty acids, thereby protecting the intestinal cells from radiation and chemotherapy-induced damage and in addition, treating other conditions such as ulcerative colitis, Crohn's disease, colonic infections and colonic motility disorders.

The aqueous solutions of the present invention can be buffered and non-buffered short chained C₂ to C₆ straight or branched chained fatty acids. Preferable such acids are selected from the group consisting of acetic acid, butyric acid, valeric acid, caproic acid, and propionic acid and mixtures thereof. It would also include other fatty acids with carbon chains ranging from C₂ to C₆ in length. These short chained fatty acids are used to emulsify lipophilic or fat-soluble anti-oxidants and other drugs. Examples of the fat-soluble anti-oxidants can be one or more members selected from the group consisting of lycopene, tococpherols (vitamin E), coenzyme Q 10 lutein and beta-carotene. Other hydrophobic drugs can be: simvastatin, fenofibrate, testosterone, haloperidol, omega 3 fatty acids, carvedilol, dronabinol, atorvastatin, itraconazole (and related compounds), isotretinoin, fentanyl, rifampin, clarithromycin, prednisone (and related corticosteroids), spironolactone, nifedipine, diazepam, and ibuprofen. The buffering agent can be any compatible system that resists a sudden change in pH such as the presence of a weak acid and a salt of the weak acid, or, a weak base and a salt of a weak base. In some instances, the buffer can be a base alone, including, but not limited to sodium hydroxide, sodium bicarbonate and potassium hydroxide.

Another aspect of the present invention relates to a method of using the present composition as an enema to prevent and treat proctitis, enteritis and colitis induced by radiation therapy and chemotherapy. The present composition may be delivered to the colon and rectum by an enema or feeding tube or other delivery systems known in the art. This mixture can be delivered prior to, during and after radiation therapy, chemotherapy and other insults likely to induce proctitis, colitis or other intestinal damage. It can also potentially be used to prevent or treat other causes of proctitis, colitis or other afflictions of the anus, rectum and colon.

### REFERENCES

1. UpToDate Online (Accessed Feb. 13, 2006).
2. Aalkjaer C. Short Chained Fatty Acids and the colon: how do they cause vasodilatation. Journal of Physiology 2002 Feb. 1; 538(Pt 3):674.
3. Naruszewicz M, Kozlowska-Wojciechowska M. Potential Parapharmaceuticals in the traditional Polish diet. Journal of Physiology and Pharmacology 2005; 56, Suppl 1, 69-79.
4. Cauza E, et al. Effects of LDL-immunoapheresis on plasma concentrations of vitamin E and carotenoids in patients with familial hypercholesterolemia. Journal of Clinical Apheresis 2004; 19(4): 174-9.
5. www.fda.gov (Accessed Feb. 22, 2006)
6. Ben-Josef E, et al. Intrarectal application of amifostine for the prevention of radiation-induced rectal injury. Seminars in Radiation Oncology 2002; 12(1 Supplement 1): 81-85.
7. Breuer R I, et al. Short chain fatty acid rectal irrigation for left-sided ulcerative colitis: a randomized, placebo controlled trial. Gut 1997; 40: 485-491.
8. Ross, M and Romrell, L. (1989). Histology. Baltimore: Williams and Wilkins.
9. Lehninger, A L, et al. (1993). Principles of Biochemistry, Second Edition. New York: Worth Publishers.
10. www.rxlist.com (accessed Apr. 14-16, 2006)

### BRIEF DESCRIPTION OF DRAWING(S)

FIG. 1 illustrates optical absorption of suspensions of beta carotene in water, acetic acid and buffered acetic acid as measured by a Beseler pm2L color analyzer.

### DETAILED DESCRIPTION OF EXAMPLE EMBODIMENT(S)

Before the present composition and method for delivery of a bioactive agent are disclosed and described, it is to be understood that this invention is not limited to the particular configurations, process steps, and materials disclosed herein as such configurations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

It must be noted that, as used in this specification and the appended claims, the singular forms “a,” “an,” and “the” include plural referents unless the context clearly dictates otherwise. Thus, for example, unless otherwise indicated reference to a buffer includes reference to two or more buffering agents, reference to “a fatty acid” includes reference to one or more of such short chained fatty acids, and reference to “a drug” includes reference to two or more of such drugs.

In describing and claiming the present invention, the following terminology will be used in accordance with the definitions set out below.

As used herein, the term “bioactive agent” or “drug” or any other similar term means any chemical or biological material or compound suitable for administration by methods previously known in the art and/or by the methods taught in the present invention and that induce desired biological or pharmacological effects, which may include but are not limited to (1) having a prophylactic effect on the organism and preventing an undesired biological effect such as preventing an infection, (2) alleviating a condition caused by a disease, for example, alleviating pain or inflammation caused as a result of disease, and/or (3) either alleviating, reducing, or completely eliminating a disease from the organism

One embodiment of the present invention relates to a composition for delivering lipophilic antioxidants to intestinal mucosa comprising: an aqueous solution of one or more short chained fatty acids and one or more lipophilic agents dissolved in said aqueous solution of one or more short chained fatty acids, wherein said composition when administered to the colon and rectum, said short-chained fatty acids are consumed by the intestinal mucosa thereby forcing said lipophilic anti-oxidants and lipophilic agents to enter the lipophilic cell membrane, whereby protecting the cells from the adverse effects of radiation and chemotherapy. The aqueous solution of short chained fatty acids allows hydrophobic agents such as lipophilic antioxidants to be dissolved or suspended in an aqueous medium (emulsification). Because the colon consumes short chained fatty acids as an energy source, the lipophilic anti-oxidants and drugs contained in the composition of the present invention become progressively less and less soluble in the aqueous medium. Therefore, when the composition of the present invention is administered to the colon, the hydrophobic anti-oxidants and drugs enter the lipophilic cell membrane driven by the intestinal consumption of the short chained fatty acids, thereby protecting the colonic cells from radiation and chemotherapy-induced damages.

The composition of the present invention can be buffered and non-buffered short chained fatty acids by a buffering agent, as described previously, such as sodium hydroxide, sodium bicarbonate and potassium hydroxide. Suitable short chained fatty acids are C₂ to C₆ straight or branched chained fatty acids. For example they can be a member selected from the group consisting of acetic acid, butyric acid, valeric acid, caproic acid and propionic acid and mixtures thereof. These short chained fatty acids are used to emulsify lipophilic or fat-soluble anti-oxidants and other drugs. Examples of the fat-soluble anti-oxidants can be one or more members selected from the group consisting of lycopene, tocopherols (vitamin E), coenzyme Q 10, lutein and beta-carotene. Other hydrophobic drugs can be: simvastatin, fenofibrate, testosterone, haloperidol, omega 3 fatty acids, carvedilol, dronabinol, atorvastatin, tacrolimus, itraconazole (and related compounds), isotretinoin, fentanyl, rifampin, clarithromycin, prednisone (and related corticosteroids), spironolactone, nifedipine, diazepam, and ibuprofen.

Another aspect of the present invention relates to a method of use the present composition as an enema to prevent and treat proctitis, enteritis and colitis induced by radiation therapy and chemotherapy. The present composition may be delivered to the colon and rectum by an enema or feeding tube or other delivery systems known in the art. This mixture can be delivered prior to, during and after radiation therapy, chemotherapy and other insults likely to induce proctitis, colitis or other intestinal damage. It can also potentially be used to prevent or treat other causes of proctitis, colitis or other afflictions of the anus, rectum and colon.

This essence of the present invention lies in the unique physiology of the colon and rectum. The colon and rectum consume short-chained fatty acids. As the short-chained fatty acids are consumed, the lipophilic anti-oxidants and other chemicals are no longer be able to remain in aqueous solution. At this time, they enter the lipophilic cell membrane, where they protect the cells from insults including, but not limited to radiation and chemotherapy. Topical application of amifostine, a hydrophilic anti-oxidant has been shown to reduce radiation-induced damage to the rectum. Because the lining of the intestines are composed of relatively rapidly-dividing cells, these cells are the most vulnerable to chemotherapy and radiation. Like amifostine, lipophilic anti-oxidants can also protect the rectum from radiation and chemotherapy-induced damage.

Unlike currently available preventative treatments for radiation and chemotherapy-induced damage to the colon, this system is unlikely to deliver significant quantities of lipophilic anti-oxidants to tissues outside of the rectum. This is again due to the unique physiology of the colon and rectum and the circulatory system. In humans, the vast majority of lipophilic anti-oxidants are carried in low-density lipoproteins. Low density lipoproteins (LDLs) are formed from materials absorbed in the small intestine, not in the large intestine. Therefore, the anti-oxidants are unlikely to be absorbed in large intestine in significant quantities to interfere with anti-tumor effects of chemotherapy and radiation therapy.

The components of the composition of the present invention are all known to be well-tolerated and safe in humans. Short chained fatty acid solutions have been delivered to the colon and rectum for treatment and prevention of a number of conditions, including radiation proctitis. Short chained fatty acids are also produced in the large intestine through natural fermentation. Anti-oxidants have also been used therapeutically for a number of conditions, including conditions of the intestines. Anti-oxidants are natural components of food, and some are vitamins essential for human health. Amifostine, an agent currently used to prevent radiation and chemotherapy-induced damage of healthy tissues in multiple organ systems, functions as an anti-oxidant. The active ingredients of the present invention are not chemically altered by the process of making. The essence of the present invention is using an aqueous short chain fatty acid solution to dissolve or suspend hydrophobic anti-oxidants or other hydrophobic drugs in an aqueous solution. When the present composition is administered to the colon and rectum where the short-chained fatty acids are consumed, the lipophilic anti-oxidants and other chemicals are no longer be able to remain in the aqueous solution therefore they are forced to enter the lipophilic cell membrane driving by the consumption of the short chained fatty acids, thereby protecting the cells from radiation and chemotherapy-induced damages.

The aqueous medium for solubilizing the lipophilic antioxidants or other lipophilic drugs used in the present invention can be an aqueous solution of short chained fatty acids with any concentrations up to and including saturated solutions. Preferably, the concentration is within a range of: acetate 60-90 mmol/liter, propionate 20-40 mmol/liter and butyrate 30-50 mmol/liter. Similar concentrations for other fatty acids and/or mixtures of fatty acids can be readily determined. The short chain fatty acids used in the present invention can be in form of salts with corresponding cations of sodium or potassium. The present composition contains a relatively high concentration of short chained fatty acids, however, it has been shown to be safe in human studies. Breuer R I, et al. Short chain fatty acid rectal irrigation for left-sided ulcerative colitis: a randomized, placebo controlled trial. Gut 1997; 40: 485-491. The pH of the composition of the present invention can be adjusted to a range of 3.5 to 10.5 and preferably about to 7-8, which is about the normal pH of human arterial blood.

Additional features and advantages of the invention will be apparent from the detailed description, taken in conjunction with the accompanying examples that follows. Reference will now be made to the exemplary embodiments illustrated, and specific language will be used herein to describe the same. It will nevertheless be understood that no limitation of the scope of the invention is thereby intended.

### EXAMPLE 1

This example illustrates how to make an aqueous solution of lipophilic antioxidants for delivering lipophilic antioxidants to intestinal mucosa.

To demonstrate the ability of short-chained fatty acids to emulsify lipophilic molecules in an aqueous solution, a number of experiments can be done.

In this experiment, three basic solutions were made:
Solution #1—Water
Solution #2—A 830 mm solution of acetic acid was made up. However, other aqueous solutions of various short-chained fatty acids at various concentrations can also be utilized.
Solution #3—Solution #2 buffered to a pH of 7.4 with sodium bicarbonate. Similarly other fatty acid solutions can be buffered to a variety of pH values using sodium bicarbonate or other buffering agents as noted above.

Each of the three solutions were heated to 90 degrees Celsius and agitated vigorously with a lipophilic anti-oxidant. In this experiment beta carotene was utilized. The concentration of beta carotene was 25,000 international units dissolved in 300 mg soybean oil. However, other lipophilic anti-oxidants such as other carotenoids including lycopene, lutein, etc. can also be utilized. These lipophilic chemicals have intense colors. The lipophilic anti-oxidants are suspended in a lipid medium such as a vegetable oil. In this instance soybean oil was utilized. Other vegetable oils that might be used include members selected from the group consisting of corn, olive rapeseed and sesame seed oils and mixtures thereof. Other lipophilic solvents including but not limited to methylene chloride, chloroform, benzene, hexane, octane, heptane could also be used to introduce the lipophilic agent to the solution.

After vigorous agitation, each solution was allowed to cool and phase separate. The solution was then pipetted from the aqueous layer and analyzed by optical absorption. Because the optical absorption coefficient for some carotenoids is well-known, the values derived would correspond to the concentration of the various lipophilic carotenoids in the solution. The composition of the solution can be altered with regard to pH and different mixtures of short chained fatty acids. A solution with a greater optical absorption means that it contains more carotenoids in the solution. In this way, the best mixtures for emulsifying various carotenoids can be determined.

On visual inspection, solution #1 was essentially transparent after the addition of beta carotene as described above. After the addition of beta carotene as described above, solution #2was slightly more intensely orange, but remained relatively translucent. After the addition of beta carotene as described above, solution #3 was intensely orange and opaque.

A Beseler pm2L color analyzer was used to examine the resultant solutions. This analyzes light intensity on a scale of 0 to 100. Higher numbers indicate increasing optical absorbance. Using the white light setting, the color analyzer was zeroed using a test tube containing water. Then, 5 samples of each of the above listed solutions were tested for optical absorbance. The results were as follows:

Solution #1 plus beta-carotene—0, 0, 1, 1, 0

Solution #2 plus beta carotene—4, 4, 5, 4, 4

Solution #3 plus beta carotene—22, 22, 22, 22, 23

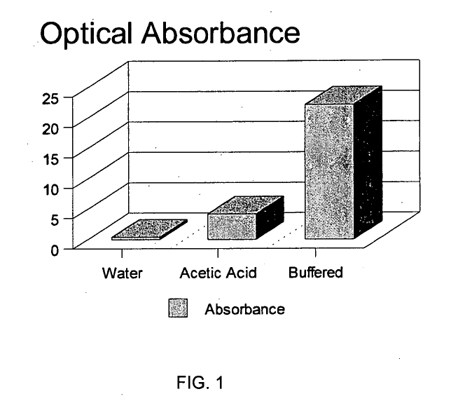

FIG. 1 illustrates optical absorption of suspensions of beta carotene in water, acetic acid and buffered acetic acid as measured by a Beseler pm2L color analyzer. This represents the average of 5 measurements of each suspension. Each solution is vigorously mixed with approximately 25,000 international units of beta carotene dissolved in soybean oil. The water solution has a pH of 6.8. The acetic acid solution is 5% acetic acid, unbuffered, with a pH of 4. The buffered solution is acetic acid 5% buffered to a pH of 7.4 using sodium bicarbonate.

These results indicate that the buffered solution of short chained fatty acids (acetic acid in this case) had more optical absorbance than the other solutions. This is a result of more beta carotene in suspension #3 than in the other suspensions.

Further experiments can use solutions 1, 2 and 3 in other ways. Lipophilic chemicals could be introduced in a similar fashion (dissolved in soybean oil and agitated with the solutions). The resulting solution can be allowed to sit, and the aqueous phase could be pipetted into flasks. From these flasks, the solution can be analyzed for concentration of the lipophilic molecules in question using mass spectroscopy. This can be used for lipophilic molecules such as vitamin E, co enzyme Q, BHA (butylated hydroxyanisol) and BHT (butylated hydroxytoluene). Again, the mixture and concentrations of short-chained fatty acids can be altered to find the most effective solution for dissolving these various chemicals.

As noted above, one of the primary functions of the fatty acids is to optimize the delivery of the lipophilic agents, such as anti-oxidants, into the intestinal mucosa membranes by means of the fatty acids being consumed. Since the fatty acids, preferably with appropriate buffering, are the solubilizers of the lipophilic agents, the concentration of fatty acids in the aqueous buffered or unbuffered solution is somewhat determinative of the concentration of lipophilic agents in that solution. Also, other factors enter in. The solubility of the lipophilic agents may vary according to the lipophilic solvent, e.g. soybean oil, etc., in which they are initially suspended or dissolved and the partitioning of the lipophilic agent from the lipophilic solvent phase into the aqueous fatty acid solution. The goal is to maximize the concentration of lipophilic agents in the aqueous fatty acid phase which is preferably buffered. Therefore, the concentraton of lipophilic agents in the aqueous fatty acid phase is best determined empirically. Any lipophilic agent concentration in the fatty acid solution is beneficial in protecting against the effects of chemo or radiation therapy in the intestinal mucosa. To that end, any concentration of lipophilic agent in the aqueous fatty acid phase, whether buffered or not, is considered to be an effective amount. Preferably the concentration of lipophilic agents in the aqueous fatty acid solution will approach saturation such that, as the fatty acids are consumed, the lipophilic agent will be readily available for entry into the mucosal lipophilic cell membranes. However, it is not practical to state exact concentrations or ranges of concentration for lipophilic agents in the fatty acid solution when there is a great degree of concentration variability that can be obtained within the scope of the teachings of this specification. One skilled in the art can readily determine optimal concentrations of the selected active lipophilic agent(s) according to the fatty acids utilized, their concentration, and the degree of buffering along with other factors taught herein.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

1. A composition for delivering lipophilic agents to intestinal mucosa comprising: an aqueous solution of one or more C₂ to C₆ fatty acids and their corresponding salts and one or more lipophilic agents dissolved in said aqueous solution of said fatty acids, wherein said composition, when administered to the colon and rectum, said fatty acids are consumed thereby forcing said lipophilic agents to enter the lipophilic cell membrane, thereby protecting the cells from the adverse effects of radiation and chemotherapy.

2. The composition according to claim 1, wherein said fatty acid is a member selected from the group consisting of butyric acid, valeric acid, caproic acid, acetic acid, propionic acid.

3. The composition according to claim 1, wherein said lipophilic agent is a member selected from the group consisting of lycopene, vitamin E, coenzyme Q 10, lutein, beta-carotene, other carotenoids, BHA and BHT.

4. The composition according to claim 1, wherein said composition has a pH value between the range of 3.5 to 10.5.

5. A method for making a composition for delivering lipophilic agents to intestinal mucosa, comprising the steps of:
1) forming a fatty acid solution by dissolving C₂ to C₆ fatty acids or corresponding salts thereof into an aqueous medium in a predetermined concentration up to a saturated solution;
2) buffering said fatty acid solution to a physiologic pH;
3) introducing an effectived amount of a lipophilic agent into the fatty acid solution;
4) agitating the resultant mixture vigorously until the suspension of the lipophilic agent has been achieved.

6. The method according to claim 5, further comprising a step of introducing an additional lipophilic agent into the solution and agitating until the suspension, of the additional lipophilic agent has been achieved.

7. The method according to claim 5, wherein said short chained fatty acid is a member selected from the group consisting of butyric acid, valeric acid, caproic acid, acetic acid, propionic acid.

8. The method according to claim 5, wherein said lipophilic agent is a member selected from the group consisting of lycopene, vitamin E, coenzyme Q 10, lutein, beta-carotene and other carotenoids, BHA and BHT.

9. The method according to claim 5, further comprising a step of adjusting the pH value of the composition to a range of between about 3.5 and 10.5, by adding a predetermined amount of a buffering agent.

10. The method according to claim 9, wherein said buffering agent is a base selected from the group consisting of potassium hydroxide, sodium hydroxide or sodium bicarbonate.

11. A method for protecting the cells from adverse effects of radiation and chemotherapy comprising delivering an effective amount of the composition of claim 1 to the colon, rectum or other lumen via enema, feeding tube or other means.

12. The method according to claim 11 wherein said effective amount is between about 1 ml and 500 ml.

13. The method according to claim 11, wherein the C₂ to C₈ fatty acid in said composition is a member selected from the group consisting of butyric acid, valeric acid, caproic acid, acetic acid, propionic acid, and corresponding salts thereof.

14. The method according to claim 11, wherein the lipophilic agent in said composition is a member selected from the group consisting of lycopene, vitamin E, coenzyme Q 10, lutein, beta-carotene and other carotenoids, BHA and BHT.

15. The method according to claim 11, wherein said composition has a pH value between the range of 3.5 and 10.5.
